(19) United States
(12) Patent Application Publication
(10) Pub. No.: US 2024/0189574 A1
(43) Pub. Date: Jun. 13, 2024

(54) Sheath with Mechanism to Stop Bleeding Around Access Site

(71) Applicant: Boston Scientific Scimed Inc., Maple Grove, MN (US)

(72) Inventors: Qian Liu, Plymouth, MN (US); Brian Reynolds, Ramsey, MN (US); Victor Kotov, St. Louis Park, MN (US)

(21) Appl. No.: 18/388,112

(22) Filed: Nov. 8, 2023

Related U.S. Application Data
(60) Provisional application No. 63/432,130, filed on Dec. 13, 2022.

(51) Int. Cl. A61M 60/865; A61M 25/00; A61M 60/135

(52) U.S. Cl. CPC A61M 60/865; A61M 25/0023; A61M 60/135; A61M 2025/0024; A61M 2202/0007

(57) ABSTRACT

A medical device delivery system includes a sheath for insertion into an access site and for use in positioning a medical device within a blood vessel. The sheath including a sheath body extending between a distal end and a proximal end. The sheath body defines a sheath lumen for receiving the medical device. A plug is arranged around the sheath body, and the plug is defined by a first disc formed of an expandable material. The first disc is configured to be positioned within the blood vessel and against a wall of the blood vessel such that the plug can seal the access site.

### CROSS-REFERENCE TO RELATED PATENT APPLICATIONS

This application claims priority to U.S. Provisional Application No. 63/432,130, filed Dec. 13, 2022, which is herein incorporated by reference in its entirety.

### TECHNICAL FIELD

The present disclosure relates to a sheath with a mechanism to stop bleeding around an access site, and more particularly, a repositioning sheath having a plug to stop bleeding around the access site.

### BACKGROUND

During processes for insertion of percutaneous circulatory devices or various other implantable medical devices, the device may be inserted into a blood vessel through an introducer sheath inserted at an access site and into the blood vessel. After placement of the device within the body, the introducer sheath may be removed and replaced with a sheath of a smaller diameter to increase blood flow through the blood vessel and thereby reduce ischemia issues for the patient. However, the sheath of a smaller diameter may allow for bleeding at the access site as the sheath size may be less than the size of the access site. There remains a need for reducing bleeding at the access site when the sheath inserted within the access site has a smaller size than the size of the access site formed by a larger sized sheath.

### SUMMARY

In Example 1, a medical device delivery system includes a sheath for insertion into an access site and for use in positioning a medical device within a blood vessel including a distal end opposite a proximal end, a sheath body extending between the distal end and the proximal end, the sheath body defining a sheath lumen for receiving the medical device, and a plug arranged around the sheath body, the plug defined by a first disc formed of an expandable material. The medical device delivery system further includes the first disc being positioned within the blood vessel and against a wall of the blood vessel, such that the plug is configured to seal the access site.

In Example 2, the medical device delivery system of Example 1 further includes the first disc being defined by a cylindrical shape.

In Example 3, the medical device delivery system of Example 1 or Example 2 further includes wherein the plug includes a second disc that is positioned adjacent the first disc and coupled to the first disc with a connector portion.

In Example 4, the medical device delivery system of Example 3 further includes wherein the first disc is defined by a first diameter and the second disc is defined by a second diameter, the first diameter being less than or equal to the second diameter.

In Example 5, the medical device delivery system of any of the preceding Examples further includes wherein the plug is composed of a braided material.

In Example 6, the medical device delivery system of any one of the preceding Examples further includes wherein the access site is defined by a diameter and the sheath is defined by a diameter, and the diameter of the sheath is less than the diameter of the access site, such that a gap is formed between the sheath and the blood vessel.

In Example 7, the medical device delivery system of Example 6 further includes wherein the plug is configured for sealing the gap formed between the sheath and the blood vessel.

In Example 8, the medical device delivery system of any one of the preceding Examples further includes wherein the medical device is a blood pump and the medical device delivery system is a blood pump delivery system.

In Example 9, a method for sealing an access site of a blood vessel includes inserting a sheath into a blood vessel, the sheath having a diameter that is less than a diameter of the access site, inserting a plug onto a proximal end of the sheath and arranging the plug around the sheath, the plug including at least a first disc, inserting the plug into the blood vessel with the plug in a collapsed configuration, and expanding the plug such that the first disc is in contact with the blood vessel to seal the access site.

In Example 10, the method of Example 9 further includes wherein when the plug is inserted into the blood vessel in the collapsed configuration, the first disc is inserted through the access site and into the blood vessel.

In Example 11, the medical device delivery system of Example 9 or Example 10 further includes wherein the plug is formed of a self-expanding material such that once the plug is inserted into the blood vessel, the first disc expands to an original position.

In Example 12, the medical device delivery system of any one of Examples 9-11 further includes wherein after expanding the plug, the first disc is arranged within the blood vessel and against the access site.

In Example 13, the medical device delivery system of any one of Examples 9-12 further includes wherein the plug further includes a second disc, and after expanding the plug, the second disc is arranged externally of the blood vessel.

In Example 14, the method of Example 13 further includes wherein the first disc and the second disc each have a cylindrical shape.

In Example 15, the method of Example 13 or Example 14 further includes wherein the first disc has a first diameter and the second disc has a second diameter, the first diameter being less than or equal to the second diameter.

In Example 16, a blood pump delivery system includes a repositioning sheath for insertion into an access site of a blood vessel and for use in positioning a blood pump within the blood vessel. The repositioning sheath further includes a distal end opposite a proximal end, a sheath body extending between the distal end and the proximal end, the sheath body defining a sheath lumen for receiving the blood pump, and a plug arranged around the sheath body and proximally to the distal end of the repositioning sheath. The plug is defined by a first disc having a cylindrical shape and the first disc is formed of an expandable material. The first disc is positioned within the blood vessel and against a wall of the blood vessel such that the plug is configured to seal the access site.

In Example 17, the blood pump delivery system of Example 16 further includes wherein the plug includes a second disc that is positioned adjacent the first disc and coupled to the first disc with a connector portion.

In Example 18, the blood pump delivery system of Example 17 further includes wherein the first disc is defined by a first diameter and the second disc is defined by a second diameter, the first diameter being less than or equal to the second diameter.

In Example 19, the blood pump delivery system of Example 16 further includes wherein the plug is composed of a braided material.

In Example 20, the blood pump delivery system of Example 19 further includes wherein the braided material is flexible such that the first disc is capable for compression.

In Example 21, the blood pump delivery system of Example 16 further includes wherein the access site is defined by a diameter and the repositioning sheath is defined by a diameter, and the diameter of the repositioning sheath is less than the diameter of the access site, such that a gap is formed between the repositioning sheath and the blood vessel.

In Example 22, the blood pump delivery system of Example 21 further includes wherein the plug is configured for sealing the gap formed between the repositioning sheath and the blood vessel.

In Example 23, a blood pump delivery system includes a repositioning sheath for insertion into an access site of a blood vessel and for use in positioning a blood pump within the blood vessel including a distal end opposite a proximal end, a sheath body extending between the distal end and the proximal end, the sheath body defining a sheath lumen for receiving the blood pump, and a plug arranged around the sheath body and proximally to the distal end of the repositioning sheath, the plug defined by a first disc having a cylindrical shape and a second disc having a cylindrical shape, the first disc and the second disc coupled by a connector portion extending between the first disc and the second disc. The first disc is positioned within the blood vessel and against a wall of the blood vessel and the second disc is positioned outside of the blood vessel, such that the plug seals the access site.

In Example 24, the blood pump delivery system of Example 23 further includes wherein the first disc has a diameter that is less than a diameter of the second disc.

In Example 25, the blood pump delivery system of Example 23 further includes wherein the first diameter has a value that is approximately equal to the value of the second diameter.

In Example 26, the blood pump delivery system of Example 24 further includes wherein the connector portion has a diameter that is less than the diameter of the first disc and the diameter of the second disc.

In Example 27, the blood pump delivery system of Example 23 further includes wherein the plug is composed of a flexible, braided material.

In Example 28, the blood pump delivery system of Example 27 further includes wherein the plug is composed of nitinol.

In Example 29, a method for sealing an access site of a blood vessel includes inserting a sheath into a blood vessel, the sheath having a diameter that is less than a diameter of the access sheath, inserting a plug onto a proximal end of the sheath and arranging the plug around the sheath, the plug including at least a first disc and a second disc, inserting the plug into the blood vessel with the plug in a collapsed configuration such that the first disc is inserted through the access site and into the blood vessel, and expanding the plug such that the first disc is in contact with the blood vessel and seals the access site.

In Example 30, the method of Example 29 further includes wherein the plug is formed of a self-expanding material such that once the plug is inserted into the blood vessel, the first disc expands to an original position.

In Example 31, the method of Example 29 further includes wherein after expanding the plug, the first disc is arranged within the blood vessel and against the access site and the second disc is arranged externally of the blood vessel.

In Example 32, the method of Example 29 further includes wherein the first disc and the second disc each have a cylindrical shape.

In Example 33, the method of Example 29 further includes wherein the first disc has a first diameter, and the second disc has a second diameter, the first diameter being less than the second diameter.

In Example 34, the method of Example 33 further includes wherein the first diameter has a value that is approximately equal to the value of the second diameter.

In Example 35, the method of Example 29 further includes wherein the plug is formed of a flexible, braided material.

### BRIEF DESCRIPTION OF THE DRAWINGS

FIG. 1 illustrates a cross sectional view of a blood vessel having an introducer sheath extending therethrough;

FIG. 2 illustrates a cross sectional view of the blood vessel having a repositioning sheath extending therethrough;

FIG. 3 illustrates a side view of a repositioning sheath having a plug arranged around the repositioning sheath;

FIG. 4 illustrates a side view of the repositioning sheath of FIG. 3 having an additional example of a plug arranged around the repositioning sheath;

FIG. 5 illustrates a perspective view of the repositioning sheath of FIG. 3 having an additional example of a plug arranged around the repositioning sheath;

FIG. 6 illustrates a perspective view of the repositioning sheath of FIG. 3 having an additional example of a plug arranged around the repositioning sheath;

FIG. 7 illustrates a side view of the repositioning sheath of FIG. 6 at least partially arranged within a representative blood vessel;

FIG. 8 illustrates a top view of the repositioning sheath of FIG. 3 having an additional example of a plug arranged around the repositioning sheath; and

FIG. 9 illustrates a top schematic view of the plug of FIG. 8.

### DETAILED DESCRIPTION

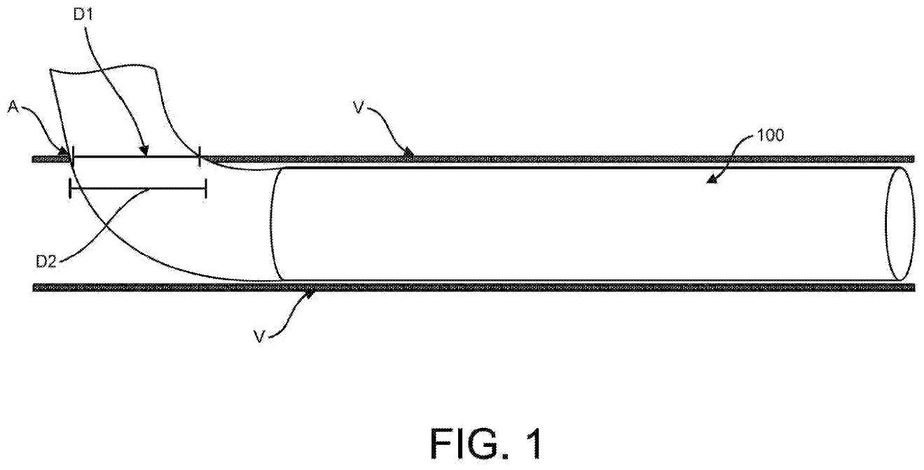

FIG. 1 illustrates a cross sectional view of a blood vessel V with an introducer sheath 100 inserted at least partially into the blood vessel V. More specifically, the introducer sheath 100 is inserted into the blood vessel through an access site A of the blood vessel V. In this way, the introducer sheath 100 has been inserted through a layer of skin of the patient and into an opening (i.e., the access site A) of the blood vessel V. In some embodiments, the introducer sheath 100 is used for facilitating the passage of various relatively large medical devices, such as a blood pump, through the introducer sheath 100 and into the blood vessel V. Hence, the introducer sheath 100 may be referred to as a large bore introducer sheath. The introducer sheath 100 is defined by a diameter D1 that may be approximately equal to a diameter D2 of the access site A. After passage of the blood pump or another type of medical device into the access site A, a smaller sheath (such as a repositioning sheath) may replace the introducer sheath 100 within the blood vessel V in order to reduce the space that is occupied within the blood vessel V by the sheath 100—thus increasing blood flow through the blood vessel V. That is, the repositioning sheath may have a smaller diameter than a diameter of the blood vessel V, ensuring that blood is able to pass around the repositioning sheath and through the blood vessel V.

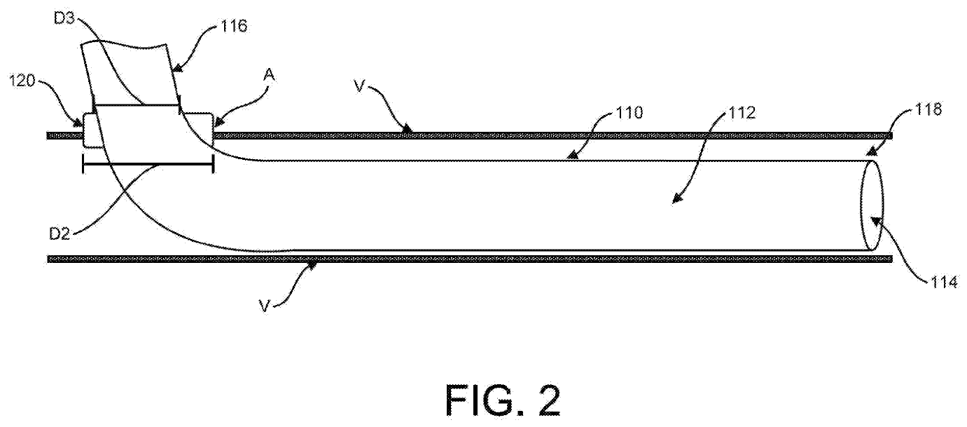

FIG. 2 illustrates an exemplary repositioning sheath 110 positioned within the blood vessel V. The repositioning sheath 110 may be defined by a generally cylindrical shape defined by an outer body 112 having a lumen 114 disposed therein. The lumen 114 may extend between a proximal end 116 and a distal end 118 of the repositioning sheath 110. The repositioning sheath 110 may be formed by various polymeric or metallic materials. In some embodiments, the repositioning sheath 110 may include an additional surface coating. The surface coating may include, but is not limited to, silicone, PET, or any other applicable polymer. As illustrated, the repositioning sheath 110 is defined by a third diameter D3. The third diameter D3 of the repositioning sheath 110 may have a value that is less than the value of the diameter D1 and/or the diameter D2. For example, in some embodiments, the third diameter D3 may range from approximately 4 mm to approximately 9 mm.

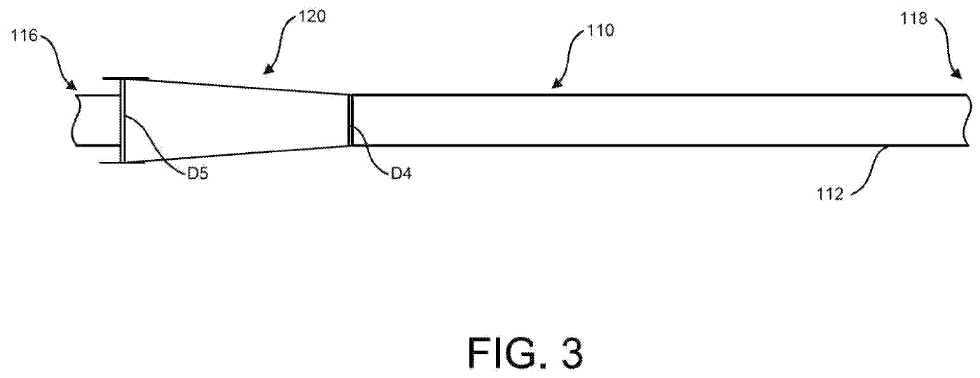

Further, as illustrated in FIG. 2, the repositioning sheath 110 may include a plug 120 that is positioned around an outer surface of the outer body 112 of the repositioning sheath 110. In other words, the plug 120 may be disposed about the circumference of the repositioning sheath 110. As will be described further herein with reference to the various embodiments of FIGS. 3-6, the plug 120 may be configured for sealing a gap at the access site A that extends between the outer body 112 of the repositioning sheath 110 and a wall, (i.e., blood vessel surface) of the blood vessel V. This gap may be formed as a result of the repositioning sheath 110 having the diameter D3 that is less than the diameter D2 of the access site A. As such, the plug 120 may be configured for providing a seal to reduce the amount of blood that may exit through the gap at the access site A between the wall of the blood vessel V and the repositioning sheath 110. In some embodiments, the plug 120 may seal the gap between the blood vessel V and the repositioning sheath 110 at the access site A and may also extend to seal against various layers of the skin of the patient that extends between the skin opening and the access site A. As will be described further herein, the plug 120 may be defined as a plug, a balloon, an expandable sheath, an expandable disc element, collar, or any other applicable structure for sealing the access site A.

FIG. 3 illustrates an enlarged portion of the repositioning sheath 110 having the plug 120 defined as a tubular structure disposed about the circumference of the repositioning sheath 110. As illustrated, the plug 120 may have a generally conical shape having an opening extending therethrough for receiving the repositioning sheath 110. In some embodiments, the plug 120 is formed of a rubber material such that the plug 120 is generally rigid in nature and will seal against a wall of the blood vessel V at the access site A. However, the plug 120 may be formed of various other materials such as silicone or low durometer polyurethane. Further, the plug 120 may have a diameter D4 and a diameter D5, wherein the diameter D4 is less than the maximum diameter D5. In some embodiments, the diameter D5 may have a value of between approximately 6 mm and approximately 9 mm. In further embodiments, the diameter D4 may have a value of between approximately 3 mm and 4 mm.

During manufacture of the repositioning sheath 110 and the plug 120, the plug 120 may be formed as a separate structure from the repositioning sheath 110 and then may be subsequently disposed onto the repositioning sheath 110. More specifically, the repositioning sheath 110 may be inserted into the blood vessel V, and then the plug 120 may be inserted over the proximal end 116 of the repositioning sheath 110 and moved along the repositioning sheath 110. Once the plug 120 is arranged onto the repositioning sheath 110, the repositioning sheath 110 may be pushed further along the repositioning sheath 110 until the plug 120 is in sealed engagement with the blood vessel V at the access site A (FIG. 1).

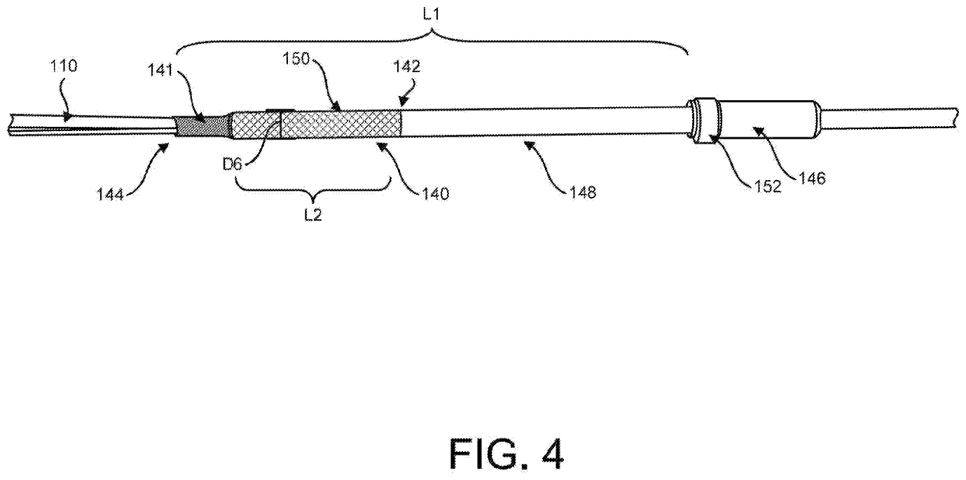

FIG. 4 illustrates an additional embodiment of a plug, illustratively an expandable plug 140. The expandable plug 140 may be a cylindrical plug disposed onto the repositioning sheath 110. The expandable plug 140 has a proximal end 142 opposite a distal end 144. The expandable plug 140 is defined by a length L1 extending therebetween. In some embodiments, the value of length L1 ranges between 13 cm and 17 cm. For example, in certain embodiments, the length L1 has a value of 15 cm. At the distal end 144, the expandable plug 140 includes a fixation means 146 that axially secures the location of the expandable plug 140 onto the repositioning sheath 110. In certain embodiments, an actuator 152 is disposed onto a distal end of the fixation means 146. As will be described further, the actuator 152 may be configured for expanding at least a portion of the expandable plug 140 for engagement with the blood vessel V at the access site A (FIG. 1).

Further, as illustrated, the expandable plug 140 has a body 148 extending between the proximal end 142 and the distal end 144. At least a portion of the body 148 is composed of a braided structure 150. The braided structure 150 may be formed of braided metal, such as a braided nitinol structure. As illustrated, the braided structure 150 is defined by a length L2 and a diameter D6. In some embodiments, the length L2 may range from approximately 2 cm and approximately 3.5 cm. In some examples, the length L2 may have a value of approximately 2.5 cm. In further embodiments, the diameter D6 may have a value ranging between approximately 15 French (Fr) to approximately 20 Fr. For example, in some embodiments, the diameter D6 has a value of approximately 15 Fr in a rest configuration and a value of approximately 20 Fr in an expanded configuration, as will be described further herein. With continued reference to FIG. 4, the expandable plug 140 includes a tapered portion 141 adjacent to the braided structure 150, which transitions from the diameter D6 of the braided structure 150 to diameter D3 of the repositioning sheath 110.

As previously described, the diameter D6 of the braided structure 150 may vary. This is due to the braided structure 150 being configured for expansion and compression through actuation of the actuator 152 of the fixation means 146. In other words, the expandable structure 150 may have a rest configuration defined by the braided structure 150 being in a longitudinally expanded state. Actuation of the actuator 152, for example rotation, or in some cases axial translation, of the actuator 152, may cause longitudinal or axial compression of the braided structure 150. In other words, the length L2 of the braided structure 150 may be reduced. As a result, and as a result of the axial positioning of the braided structure 150 being secured by the fixation means 146, the diameter D6 of the braided structure 150 will increase. As such, actuation of the actuator 152 may cause the braided structure to expand into sealed engagement with a wall of the blood vessel V surrounding the access site A.

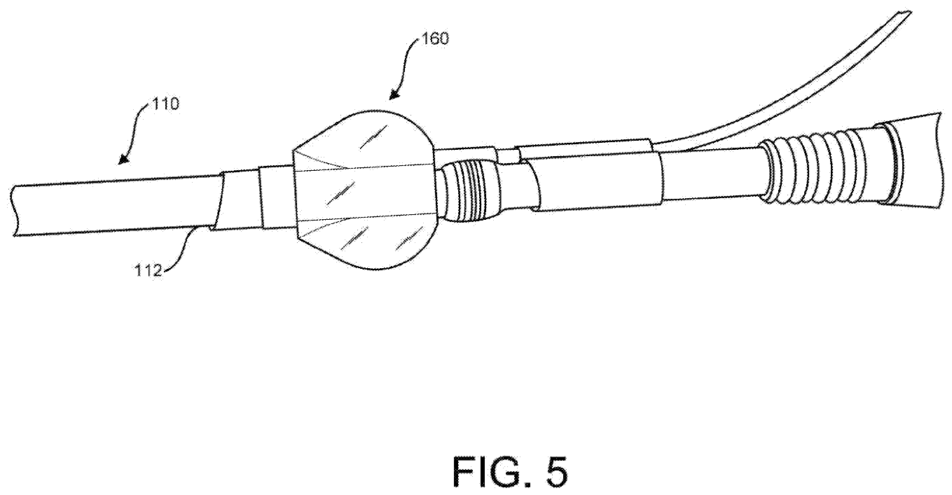

FIG. 5 illustrates an additional embodiment of a plug for use with the repositioning sheath 110, illustratively, a balloon 160. As illustrated, the balloon 160 may be disposed around the outer body 112 of the repositioning sheath 110. The balloon 160 may be formed of a semi-compliant and/or compliant material such that it may be inflated and deflated upon actuation of the balloon 160. For example, the balloon 160 may be arranged onto the repositioning sheath 110 in a first, collapsed configuration prior to insertion of the repositioning sheath 110 into the access site A. After positioning of the repositioning sheath 110 into the access site A, the balloon 160 may be inflated such that the balloon 160 comes into contact with a wall of the blood vessel V to seal the access site A.

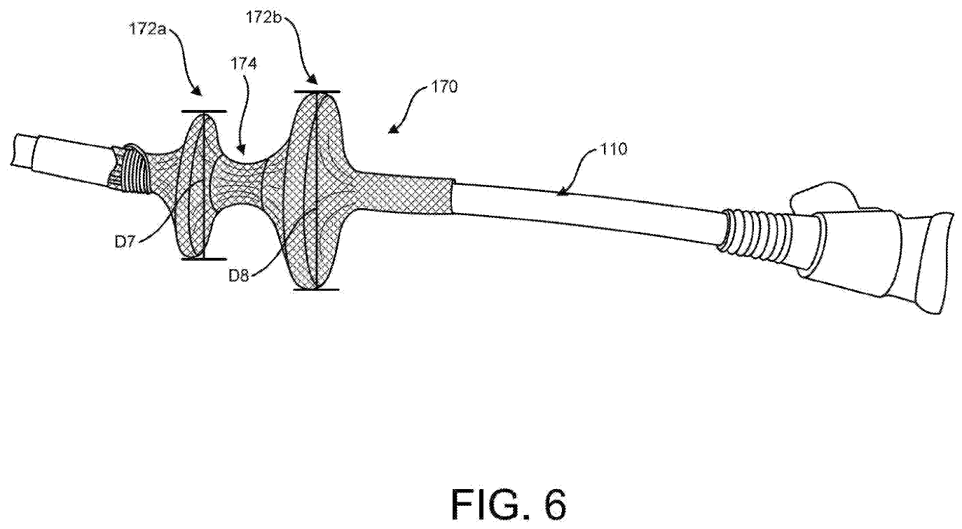

FIG. 6 illustrates an additional embodiment of a plug for use with the repositioning sheath 110, illustratively an expandable disc element 170. More particularly, the expandable disc element 170 is arranged around the outer body 112 of the repositioning sheath 110. In embodiments, the expandable disc element 170 has at least one disc element 172. In some embodiments, the expandable disc element 170 may have a first disc 172a and a second disc 172b and a connector portion or a waist portion 174 extending therebetween and coupling the first and second discs 172a, 172b. As illustrated, the first disc 172a may have a diameter D7 while the second disc 172b may have a diameter D8. In some embodiments, the diameter D7 is less than the diameter D8. In other embodiments, the diameter D7 may be approximately equal to or greater than the diameter D8. In these embodiments, one of the first disc 172a and the second disc 172b may be positioned against a wall of the blood vessel V at the access site A to reduce the bleeding at the access site A. For example, the first disc 172a may be positioned within the blood vessel V and against an inner surface of the blood vessel V at the access site A, while the second disc 172b may be positioned outside of the blood vessel V and against an outer surface of the blood vessel V at access site A. However, in various other embodiments, the expandable disc element 170 may only include the first disc 172a positioned within the blood vessel V and against an inner surface of the blood vessel V at the access site A. In further embodiments, the expandable disc element 170 may include any number of discs.

The expandable disc element 170 may be composed of a flexible, braided material. For example, in some embodiments, the expandable disc element 170 is formed of nitinol. In this way, when the expandable disc element 170 is inserted onto the repositioning sheath 110 and inserted into the access site A (FIG. 1), the first disc 172a may be in a compressed configuration wherein the first disc 172a is flattened onto the repositioning sheath 110 and once the first disc 172a is positioned within the blood vessel V, the first disc 172a may actuated to expand back out into a rest configuration and be positioned against the blood vessel V. In some embodiments, insertion of the expandable disc element 170 into the access site A is facilitated by tension being applied onto the first disc 172a to collapse the first disc 172a such that the diameter D7 may have a reduced value. In this way, the first disc 172a may be inserted into the blood vessel V. When the first disc 172a has been fully inserted into the blood vessel V, the tension may be released and the first disc 172a may expand back to the original positioning.

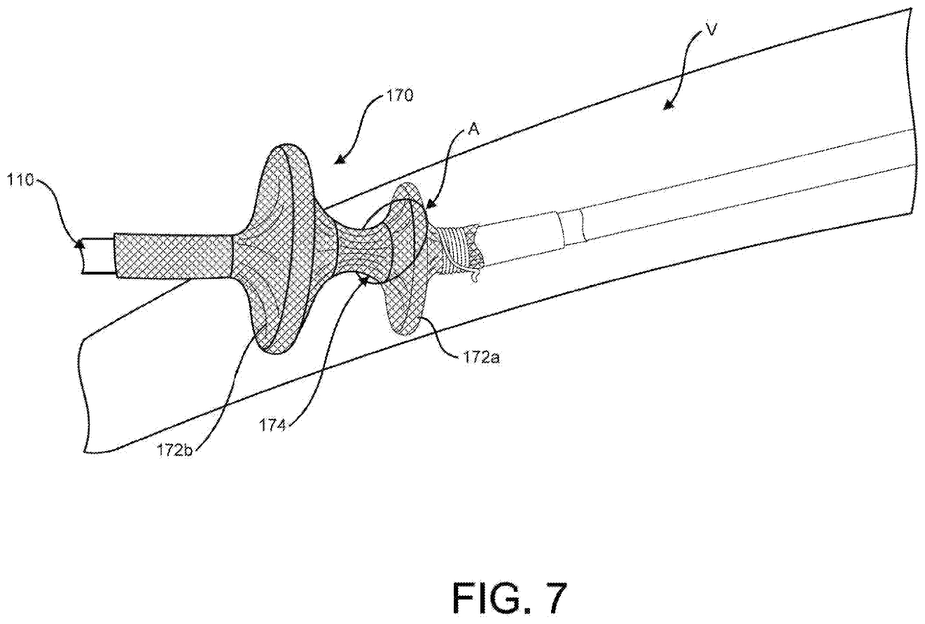

FIG. 7 illustrates the embodiment of FIG. 6 with a portion of the expandable disc element 170 arranged around the outer body 112 of the repositioning sheath 110 and the repositioning sheath 110 at least partially disposed within a schematic representation of the blood vessel V. More particularly, the first disc 172a is arranged within the blood vessel V while the second disc 172b remains positioned externally to the blood vessel V. In this way, repositioning sheath 110 may be adjusted (for example, pulled proximally) once inserted at least partially into the blood vessel V until the first disc 172a is placed against an inner surface of the blood vessel V at the access site A and the second disc 172b is positioned on the outer surface of the blood vessel V around the access site A.

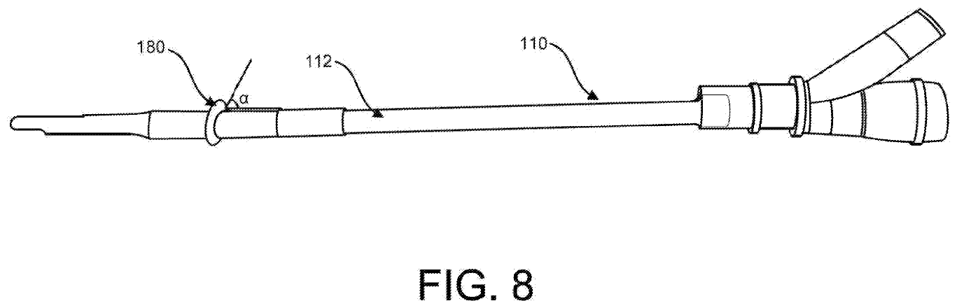

Further, FIG. 8 illustrates an additional embodiment of a plug that may be used in combination with the repositioning sheath 110. For example, a flexible collar 180 is illustrated as arranged around the outer body 112 of the repositioning sheath 110. More specifically, the flexible collar 180 is arranged around an entire circumference of the repositioning sheath 110. In some embodiments, the flexible collar 180 is arranged around the repositioning sheath 110 such that the flexible collar 180 is angled relative to the longitudinal axis L. For example, the flexible collar 180 may extend at an angle α relative to the longitudinal axis L of the repositioning sheath 110. After positioning of the repositioning sheath 110 into the access site A, the collar 180 may be positioned such that the collar 180 comes into contact with a wall of the blood vessel V to seal the access site A. For example, the collar 180 may be inserted at least partially into the blood vessel V until the collar 180 is placed against an inner surface of the blood vessel V at the access site A to seal the access site A. In other embodiments, the collar 180 may be placed against an outer surface of the blood vessel V at the access site to seal the access site A.

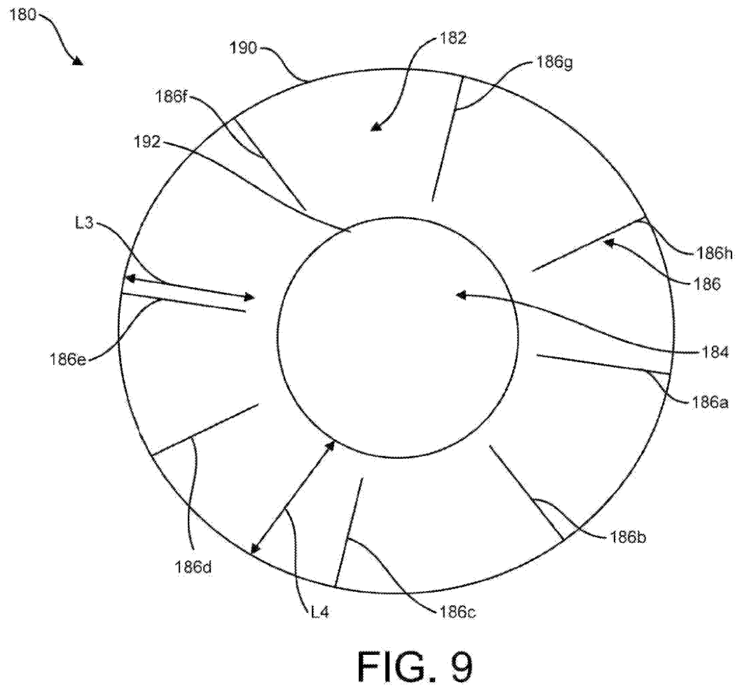

A schematic top view of the flexible collar 180 is shown in FIG. 9. As illustrated, the flexible collar 180 has a generally circular configuration defined by a main body 182 and a lumen 184 extending therethrough. The lumen 184 is illustrated as being generally circular in shape. This allows for the flexible collar 180 to engage with the outer body 112 of the repositioning sheath 110 to provide a seal. Further, while the main body 182 is illustrated as having a generally circular configuration, the main body 182 may have varying shape profiles.

Further, in some embodiments, the main body 182 may include a plurality of slits 186 extending within the main body 182. More particularly, the main body 182 is defined by an outer surface 190 and an inner surface 192 defining the lumen 184. The plurality of slits 186 are illustrated as extending from the outer surface 190 at least partially into the main body 182 towards the inner surface 192. For example, the plurality of slits 186 may extend a length L3 that is approximately 50% of a length L4 extending between the outer surface 190 and the inner surface 192 of the main body 182. In some embodiments, the length L3 may have a value of between approximately 25% and approximately 75%. For example, the value of length L3 may have a value ranging between approximately 1 mm and approximately 2 mm. The value of the length L4 may have a value ranging between approximately 2 mm and approximately 4 mm. In the illustrative embodiment of FIG. 9, the plurality of slits 186 includes seven slits 186a-186g, however, any number of slits may be incorporated. The plurality of slits 186 may increase the ease with which that the repositioning sheath 110 enters and exits the blood vessel V. For example, the plurality of slits 186 may provide for flexibility to facilitate insertion of the repositioning sheath 110 while reducing the chances of damage of the blood vessel V. Additionally, the main body 182 of the flexible collar 180 may be composed of a flexible polymer such that the flexible collar 180 maintains flexible to accommodate varying environments and reduce damage of any tissue that the flexible collar 180 comes into contact with. For example, the flexible collar 180 may be composed of silicone or low durometer polyurethane.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the above-described features.

We claim:

1. A blood pump delivery system comprising:
a repositioning sheath for insertion into an access site of a blood vessel and for use in positioning a blood pump within the blood vessel, the repositioning sheath comprising:
a distal end opposite a proximal end,
a sheath body extending between the distal end and the proximal end, the sheath body defining a sheath lumen for receiving the blood pump, and
a plug arranged around the sheath body and proximally to the distal end of the repositioning sheath, the plug defined by a first disc having a cylindrical shape, the first disc is formed of an expandable material,
wherein the first disc is configured to be positioned within the blood vessel and against a wall of the blood vessel such that the plug can seal the access site.

2. The blood pump delivery system of claim 1, wherein the plug includes a second disc that is positioned adjacent the first disc and coupled with a connector portion.

3. The blood pump delivery system of claim 2, wherein the first disc is defined by a first diameter and the second disc is defined by a second diameter, the first diameter being less than or equal to the second diameter.

4. The blood pump delivery system of claim 1, wherein the plug is composed of a braided material.

5. The blood pump delivery system of claim 4, wherein the braided material is flexible such that the first disc is compressible.

6. The blood pump delivery system of claim 1, wherein the repositioning sheath is defined by an outer sheath diameter, wherein the outer sheath diameter is less than a diameter of the blood vessel such that a gap is formed between the repositioning sheath and the blood vessel.

7. The blood pump delivery system of claim 6, wherein the plug is sized for sealing the gap formed between the repositioning sheath and the blood vessel.

8. A blood pump delivery system comprising:
a repositioning sheath for insertion into an access site of a blood vessel and for use in positioning a blood pump within the blood vessel, the repositioning sheath comprising:
a distal end opposite a proximal end,
a sheath body extending between the distal end and the proximal end, the sheath body defining a sheath lumen for receiving the blood pump, and
a plug arranged around the sheath body and proximally to the distal end of the repositioning sheath, the plug defined by a first disc having a cylindrical shape and a second disc having a cylindrical shape, the first disc and the second disc coupled by a connector portion extending between the first disc and the second disc,
wherein the first disc is configured to be positioned within the blood vessel and against a wall of the blood vessel,
wherein the second disc is configured to be positioned outside of the blood vessel such that the plug seals the access site.

9. The blood pump delivery system of claim 8, wherein the first disc has a diameter that is less than a diameter of the second disc.

10. The blood pump delivery system of claim 8, wherein the first disc has a diameter that is approximately equal to a diameter of the second disc.

11. The blood pump delivery system of claim 8, wherein the connector portion has a diameter that is less than both a diameter of the first disc and a diameter of the second disc.

12. The blood pump delivery system of claim 8, wherein the plug comprises a flexible, braided material.

13. The blood pump delivery system of claim 8, wherein the plug comprises nitinol.

14. A method comprising:
inserting a sheath into a blood vessel, the sheath having a diameter that is less than a diameter of an access sheath used to initially access the blood vessel at an access site;
inserting a plug onto a proximal end of the sheath and arranging the plug around the sheath, the plug including at least a first disc and a second disc;
inserting the plug into the blood vessel while the plug is in a collapsed configuration such that the first disc is inserted through the access site and into the blood vessel; and
expanding the plug such that the first disc is in contact with the blood vessel and seals the access site.

15. The method of claim 14, wherein the plug is formed of a self-expanding material such that, once the plug is inserted into the blood vessel, the first disc expands to an original configuration that is larger than the collapsed configuration.

16. The method of claim 14, wherein after expanding the plug, the first disc is arranged within the blood vessel and against the access site and the second disc is arranged externally to the blood vessel.

17. The method of claim 14, wherein the first disc and the second disc each have a cylindrical shape.

18. The method of claim 14, wherein the first disc has a first diameter, and the second disc has a second diameter, the first diameter being less than the second diameter.

19. The method of claim 14, wherein the first disc has a first diameter, and the second disc has a second diameter, the first diameter being approximately equal to the second diameter.

20. The method of claim 14, wherein the plug comprises a flexible, braided material.
